(19) United States
(12) Patent Application Publication
(10) Pub. No.: US 2013/0182220 A1
(43) Pub. Date: Jul. 18, 2013

(54) IMAGE FORMING METHOD AND IMAGE FORMING APPARATUS

(71) Applicant: CANON KABUSHIKI KAISHA, Tokyo (JP)

(72) Inventor: Takashi Naba, Kawasaki-shi (JP)

(73) Assignee: CANON KABUSHIKI KAISHA, Tokyo (JP)

(21) Appl. No.: 13/737,196

(22) Filed: Jan. 9, 2013

(30) Foreign Application Priority Data
Jan. 16, 2012 (JP) 2012-006007

(51) Int. Cl. A61B 3/10

(52) U.S. Cl. CPC A61B 3/102
USPC 351/206; 351/246

(57) ABSTRACT

Provided is an image forming method of forming an object image by combining a plurality of tomographic images acquired by using an optical coherence tomographic method, including: acquiring, within a first predetermined period, a first three-dimensional image of a first area including a characteristic portion of the object and first tomographic images as a part of the plurality of tomographic images of a second area different from the first area; acquiring, within a second predetermined period, a second three-dimensional image of the first area and second tomographic images as a part of the plurality of tomographic images of the second area, the second tomographic images being different from the first tomographic images; and aligning positions of the first tomographic images and the second tomographic images by using, as references, the characteristic portion included in the first three-dimensional image and the characteristic portion included in the second three-dimensional image.

### BACKGROUND OF THE INVENTION

1. Field of the Invention

The present invention relates to an image forming method and an image forming apparatus employing an optical coherence tomographic method, and more particularly, to an image forming method using an optical coherence tomography imaging apparatus which includes a coherence optical system and is used in an ophthalmic care, and to an image forming apparatus suitable for implementing the image forming method.

2. Description of the Related Art

Currently, there are used various types of ophthalmological apparatus using an optical apparatus.

For instance, as an optical apparatus for observing an eye, there are used various apparatus such as an anterior eye imaging apparatus, a fundus camera, and a confocal scanning laser ophthalmoscope (SLO). Of those, an optical coherence tomography (OCT) imaging apparatus employing an optical coherence tomographic method (hereinafter referred to as an OCT apparatus) is an apparatus capable of acquiring a tomographic image of a sample with high resolution. For this reason, the OCT apparatus is becoming an indispensable apparatus as an ophthalmological apparatus for retina-specialized outpatient care. The above-mentioned OCT apparatus is an apparatus which performs high sensitivity analysis and measurement by projecting low coherence light onto a sample and splitting reflection light from the sample using a coherence system. In addition, the OCT apparatus is capable of acquiring a tomographic image with high resolution by scanning the sample with the low coherence light. This enables the OCT apparatus to acquire a tomographic image of a retina in the fundus of an eye to be inspected with high resolution, and hence the OCT apparatus is used widely for ophthalmological diagnosis of retina or the like.

In the conventional technology for acquiring a three-dimensional image, for example, in an ultrasonographic apparatus disclosed in Japanese Patent Application Laid-Open No. 2002-153473, a three-dimensional image acquired from a first scan in a first scanning direction and a three-dimensional image acquired from a second scan in a second scanning direction opposite to the first scanning direction are superimposed to acquire a higher-resolution three-dimensional image.

Further, in Japanese Patent Application Laid-Open No. H08-294485, when superimposing time-series computed tomography (CT) images of the same portion of the same examinee for radiologic interpretation, alignment of the images is performed by a two-dimensional correlation operation of the tomographic images.

As described above, a three-dimensional retina image acquired by using the OCT apparatus is considerably useful in observation of a disease of the eye. When acquiring a three-dimensional image of a necessary portion of a retina with a sufficient resolution, for example, in order to acquire a retina image of 2 mm×2 mm in the X and Y directions and 2 mm in the Z direction that is a thickness direction of the retina (a thickness of the retina is about 0.5 mm) with a resolution of 5 μm, it is required to acquire 400 tomographic images of B-scan each including 400 pixels in the X direction and 400 pixels in the Z direction. The tomographic image of B-scan is acquired by performing a plurality of times of A-scan (scanning in the Z direction) in the X direction.

At this time, for example, it takes 10 μsec or longer to acquire a tomographic image of A-scan. Therefore, it takes 4 msec or longer to acquire a tomographic image of B-scan. Accordingly, a period of time required to acquire a three-dimensional tomographic image is 1.6 sec or longer.

In addition, as illustrated in FIG. 6, the eye shows a movement in one direction, which is called “drift” (moving angle of 2 minutes of arc to 5 minutes of arc), a fine movement, which is called “tremor (fixation fine movement)” (moving angle of 50 seconds of arc to 60 seconds of arc), and an abrupt movement with less frequency, which is called “flick” (moving angle of 2 minutes of arc to 15 minutes of arc). Further, the eye constantly moves by a movement of a body of the examinee, and hence, when the three-dimensional image is acquired for a period of time as long as 1.6 sec or longer, it may cause a problem that the image is distorted.

Regarding the drift and the tremor that cause the distortion of the acquired image due to the movement of the examinee, a less distorted three-dimensional image can be acquired within data by acquiring the three-dimensional image within a period of time sufficiently shorter than the period of time of the movement illustrated in FIG. 6, for example, within 0.1 sec. However, it takes 4 msec or longer to acquire a single image of B-scan as described above, only 25 images of B-scan can be acquired within 0.1 sec. For this reason, in order to acquire a three-dimensional image as illustrated in FIG. 7, the method disclosed in Japanese Patent Application Laid-Open No. 2002-153473 can be considered, in which a plurality of rough images acquired within 0.1 sec are superimposed as illustrated in FIG. 8. In this case, the rough images are shifted from one another due to the movement of the examinee, and hence alignment of the images is required as disclosed in Japanese Patent Application Laid-Open No. H08-294485. However, the rough images in this case are acquired from different portions from one another, and hence superimposing the images by aligning images of the same portion cannot be performed.

In addition, even when the images are acquired within 0.1 sec, if there is an abrupt movement, which is called the “flick”, during the acquisition of the images, the data cannot be used due to distortion in the image, and therefore, it is required to detect whether or not the flick is generated during the acquisition of the images.

### SUMMARY OF THE INVENTION

The present invention has been made in view of the above-mentioned circumstances, and an object of the present invention is to provide an image forming method which is capable of acquiring a high-resolution three-dimensional retina image without influencing the image by a fixation fine movement or a movement of a body of an examinee, and to provide an image forming apparatus suitable for implementing the image forming method.

In order to achieve the above-mentioned object, according to an exemplary embodiment of the present invention, there is provided an image forming method of acquiring a plurality of tomographic images of an object to be inspected by using an optical coherence tomographic method and forming a three-dimensional image of the object based on the plurality of acquired tomographic images, the image forming method including; acquiring, within a first predetermined period, a first three-dimensional image of a first area including a characteristic portion of the object and first tomographic images as a part of the plurality of tomographic images of a second area different from the first area, acquiring, within a second predetermined period, a second three-dimensional image of the first area and second tomographic images as a part of the plurality of tomographic images of the second area, the second tomographic images being different from the first tomographic images, and aligning positions of the first tomographic images and the second tomographic images by using, as references, the characteristic portion included in the first three-dimensional image and the characteristic portion included in the second three-dimensional image.

According to the present invention, in an optical coherence tomography (OCT) imaging apparatus and particularly in an image forming apparatus including the OCT apparatus for acquiring a tomographic image of a retina in a fundus of an eye to be inspected, a retina image can be acquired without being influenced by a fixation fine movement or a movement of a body of the examinee when acquiring a high-resolution three-dimensional retina image.

Further features of the present invention will become apparent from the following description of exemplary embodiments with reference to the attached drawings.

### BRIEF DESCRIPTION OF THE DRAWINGS

FIG. 1A is a diagram illustrating an optical system of an OCT apparatus according to an embodiment of the present invention.

FIG. 1B is a diagram illustrating another optical system of the OCT apparatus according to the embodiment of the present invention.

FIG. 2 is a diagram illustrating a representative fundus image acquired by two-dimensionally imaging a fundus.

FIG. 3A is a flowchart illustrating a flow of an operation according to the embodiment of the present invention.

FIG. 3B is a flowchart illustrating a flow of an operation executed in pre-scanning of the flow illustrated in FIG. 3A.

FIG. 4 is a diagram illustrating a representative rough image in the Y direction acquired according to the embodiment of the present invention.

FIG. 5 is a flowchart illustrating a flow of an evaluation on a plane according to the embodiment of the present invention.

FIG. 6 is a diagram illustrating a movement of an eye.

FIG. 7 is a diagram illustrating a target three-dimensional image.

FIG. 8 is a diagram illustrating a rough image in the Y direction acquired within 0.1 sec.

### DESCRIPTION OF THE EMBODIMENTS

### Embodiment

The present invention can be particularly applied to a Fourier domain (FD)-OCT apparatus for high speed imaging.

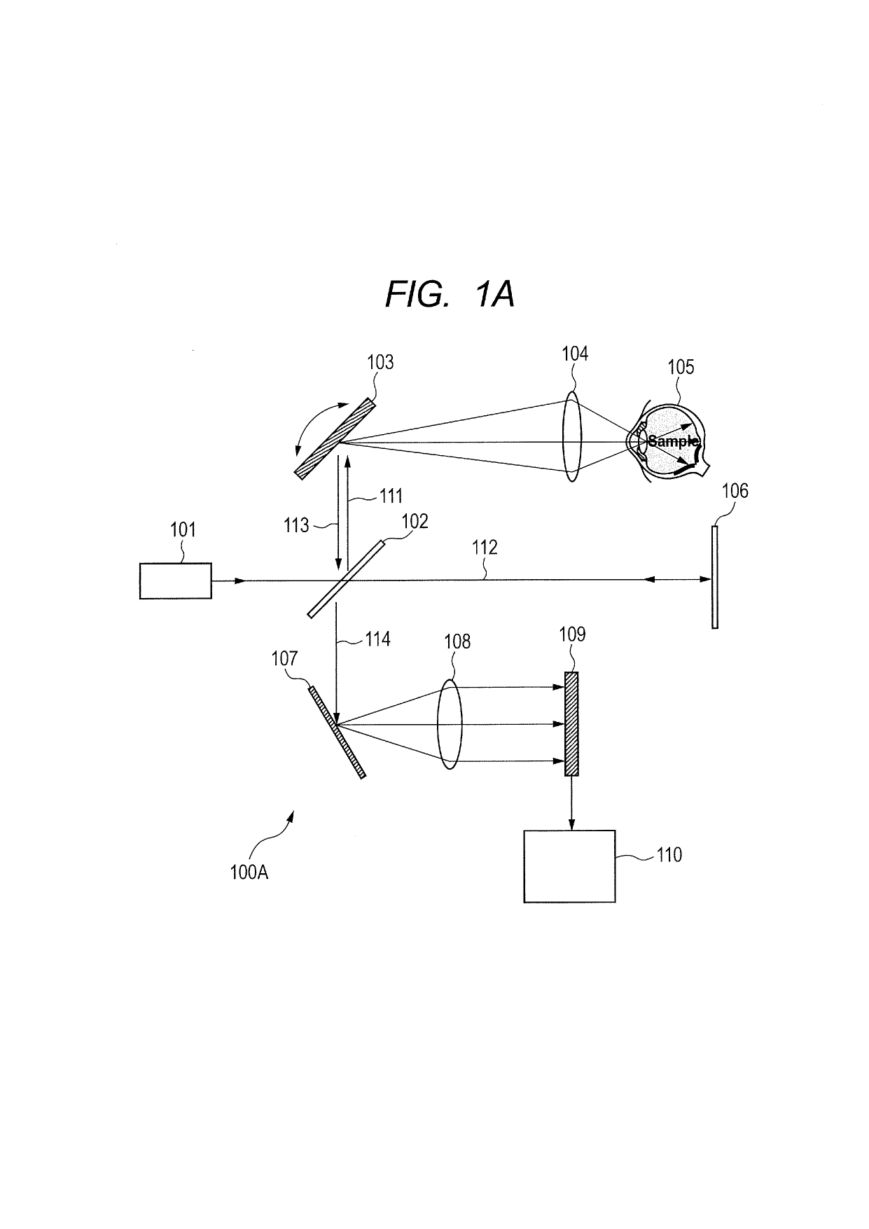

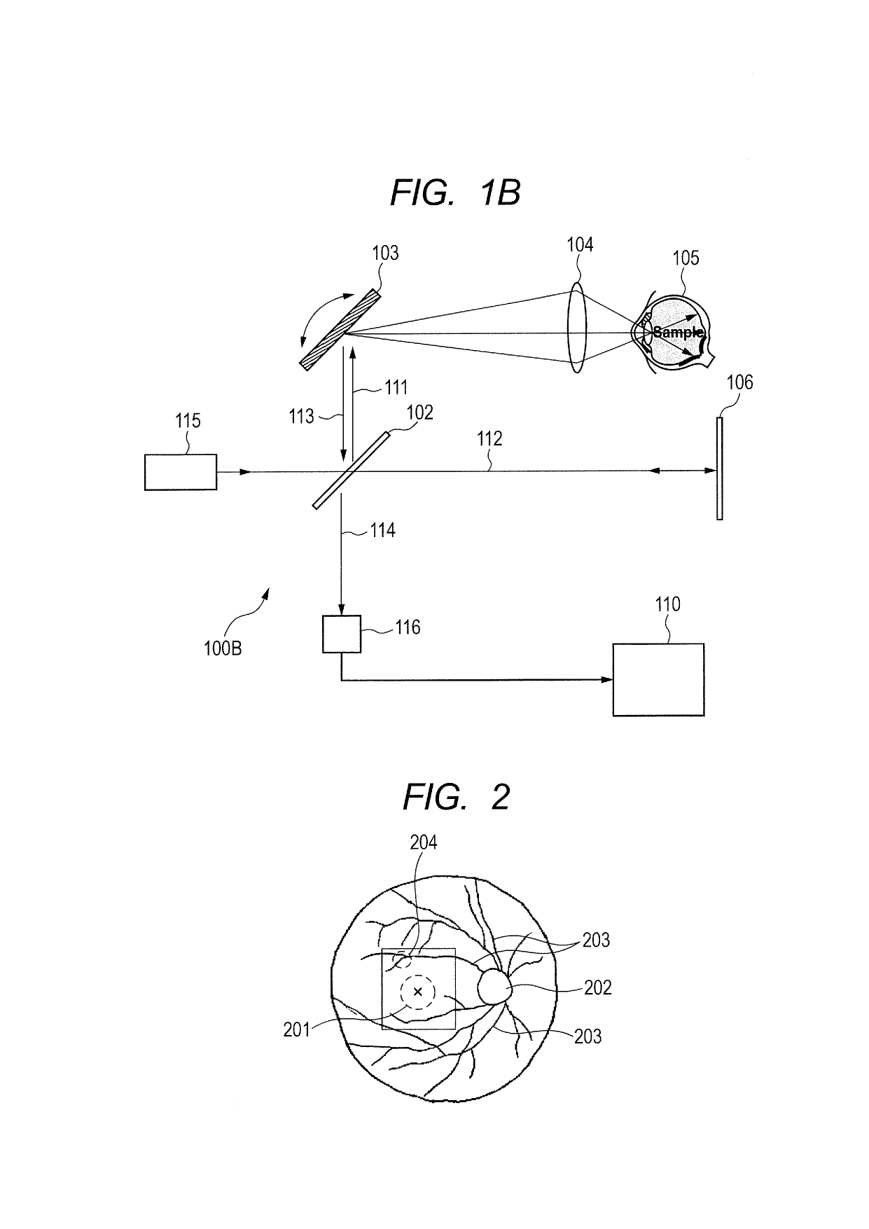

The FD-OCT apparatus can be roughly divided into a spectral domain (SD)-OCT apparatus and a swept source (SS)-OCT apparatus. Although a fundus (retina) of an eye to be inspected is described as an example of an object to be inspected, the present invention is not limited to any particular target. For example, the object to be inspected can be a skin or an organ of the examinee. In this case, the present invention can be applied to a medical apparatus such as an endoscope in addition to the ophthalmological apparatus. Firstly, an overall configuration of the SD-OCT apparatus is broadly described. FIG. 1A is a schematic diagram of an SD-OCT apparatus 100A.

Light emitted from a light source 101 is split into measuring light 111 and reference light 112 by a beam splitter 102. The measuring light 111 is returned as return light 113 by reflection and scattering at an eye 105 that is a target to be observed, and then combined with the reference light 112 by the beam splitter 102 to produce interference light 114. The interference light 114 is dispersed by a diffraction grating 107, and then imaged on a line sensor 109 by a lens 108. Each output of the line sensor 109 is subjected to Fourier transform with a position in the line sensor, i.e., a wave number of the interference light to acquire a tomographic image of the eye 105 by a control unit (CPU 110). The CPU 110 performs driving control of a scanner, a reference mirror, and the like and each process for generating a three-dimensional image, which are described later, by using corresponding modules.

Next, the light source 101 and matters relevant thereto are described. The light source 101 is a super luminescent diode (SLD), which is a typical low coherence light source. The light source 101 has a wavelength of 830 nm and a bandwidth of 50 nm. Here, the bandwidth is an important parameter because the bandwidth influences the resolution of the acquired tomographic image in the optical axis direction. In addition, the light source of an SLD type is used in this embodiment, but an amplified spontaneous emission (ASE) type or the like may also be used as long as the light source emits low coherence light. In addition, concerning the wavelength of light, near-infrared light is suitable because the light is used for measuring an eye. Further, because the wavelength influences the resolution of the acquired tomographic image in the lateral direction, the wavelength is desirably as short as possible. Here, the wavelength is 830 nm. Depending on the measurement site to be observed, another wavelength may be selected.

Next, an optical path of the reference light 112 is described. The reference light 112 split by the beam splitter 102 is reflected at a mirror 106 and returns to the beam splitter 102.

By setting an optical path length of the reference light 112 equal to that of the measuring light 111, the measuring light 111 and the reference light 112 can interfere with each other.

Next, an optical path of the measuring light 111 is described. The measuring light 111 split by the beam splitter 102 enters a mirror of an XY scanner 103. Here, the XY scanner 103 is described as a single mirror for simple description, but the XY scanner 103 actually has two mirrors of an X scan mirror and a Y scan mirror disposed closely to each other so as to raster-scan the retina on the eye 105 in a direction perpendicular to the optical axis. Further, adjustment is performed so that the center of the measuring light 111 is aligned with the rotation center of the mirror of the XY scanner 103. The measuring light 111 is focused on the retina by a lens 104. With this optical system, the measuring light 111 entering the eye 105 becomes the return light 113 by reflection and scattering at the retina of the eye 105.

In addition, the OCT apparatus generally includes A-scan laser ophthalmoscope (SLO) (not shown) or an optical system for two-dimensionally acquiring a fundus image (not shown), in order to monitor an imaging position.

A spectroscopic system is described below. As described above, the interference light 114 is dispersed by the diffraction grating 107. This spectroscopy is performed under the same wavelength condition as the center wavelength and the bandwidth of the light source. In addition, the line sensor 109 for measuring the interference light generally is a line sensor of a CCD type or a CMOS type, and both of the types provide substantially the same result.

FIG. 1B is a schematic diagram of an SS-OCT apparatus 100B. The SS-OCT apparatus is different from the SD-OCT apparatus in that the light source is changed from a low coherence light source having a bandwidth to a light source (swept source) 115 configured to scan the wavelength of light, and the light receiving portion is changed from the spectroscope to a simple light receiving element 116. That is, in the SD-OCT, the light from the light source having the bandwidth is dispersed at the light receiving portion. However, in the SS-OCT, signals similar to those of the line sensor 109 can be acquired by scanning the wavelength of the light source and detecting the interference light in synchronization with the scanning of the wavelength.

The light source 115 of the SS-OCT apparatus can be implemented by inserting, in a ring-type fiber laser cavity, a mirror cavity that is capable of changing a cavity length in a very small amount, and the light receiving element 116 can be implemented with a PIN photodiode.

In the OCT apparatus described above, when a measurement is performed without moving the XY scanner 103, an image of A-scan can be acquired from an output of the Fourier transform. For each end of the A-scan, the scanner is continuously moved in the X direction by an amount of the resolution and the scanning results are combined to form a tomographic image, with the result that an image of B-scan can be acquired. In the same manner, for each end of the B-scan, the scanner is moved in the Y direction and the scanning results are combined to form an image, with the result that a three-dimensional retina image can be acquired.

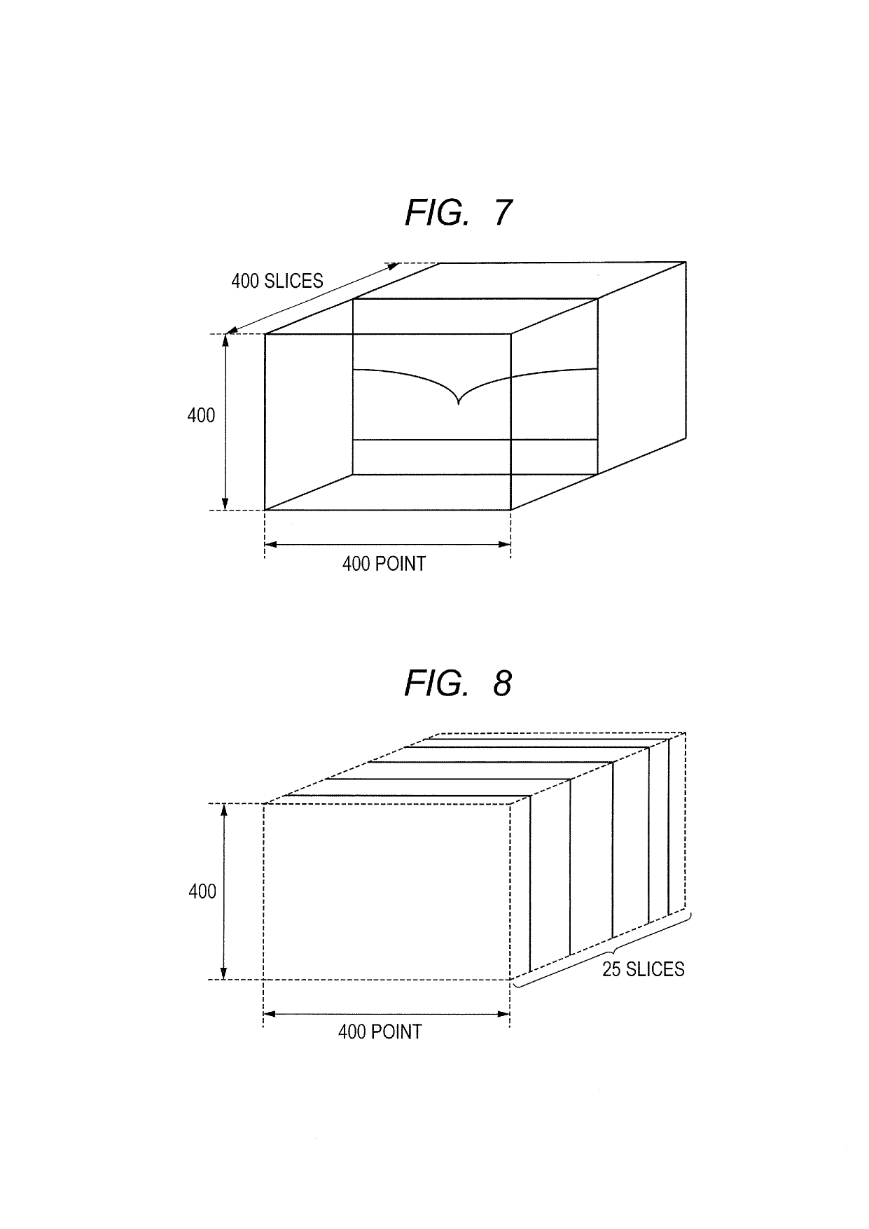

At this time, a fine scanning amount in the Y direction provides a fine three-dimensional image as illustrated in FIG. 7, and a rough scanning amount in the Y direction provides a rough three-dimensional image in the Y direction as illustrated in FIG. 8 (hereinafter referred to simply as a rough three-dimensional image).

The center of imaging is set by placing a bright spot, which is generally called “fixation lamp”, on an optical axis and causing the examinee to stare at the fixation lamp so that a macula that is the center of the field of view can be placed on the optical axis and the scanning can be performed focusing around the macula.

FIG. 2 illustrates a representative fundus image acquired by two-dimensionally imaging a fundus. The fundus image is displayed on a display unit such as a monitor arranged in association with the CPU 110. As illustrated in FIG. 2, the fundus image shows a macula portion 201 that is the center of the field of view, an optic disk portion 202 where optic nerves are concentrated, and blood vessels 203. As a feature of the macula portion, photoreceptor cells are densely concentrated, and hence there is no blood vessel in the macula portion, which is conspicuous at the center fovea in the center portion, where most lesions related to the eyesight are observed. For this reason, the imaging of the OCT is focused on the macula portion and the optic disk portion for diagnosing the glaucoma. A branch point of the blood vessel such as an area 204 is suitable for a reference portion in the present invention. The blood vessel has roughly the same pattern regardless of individual, and hence the blood vessel can be easily targeted.

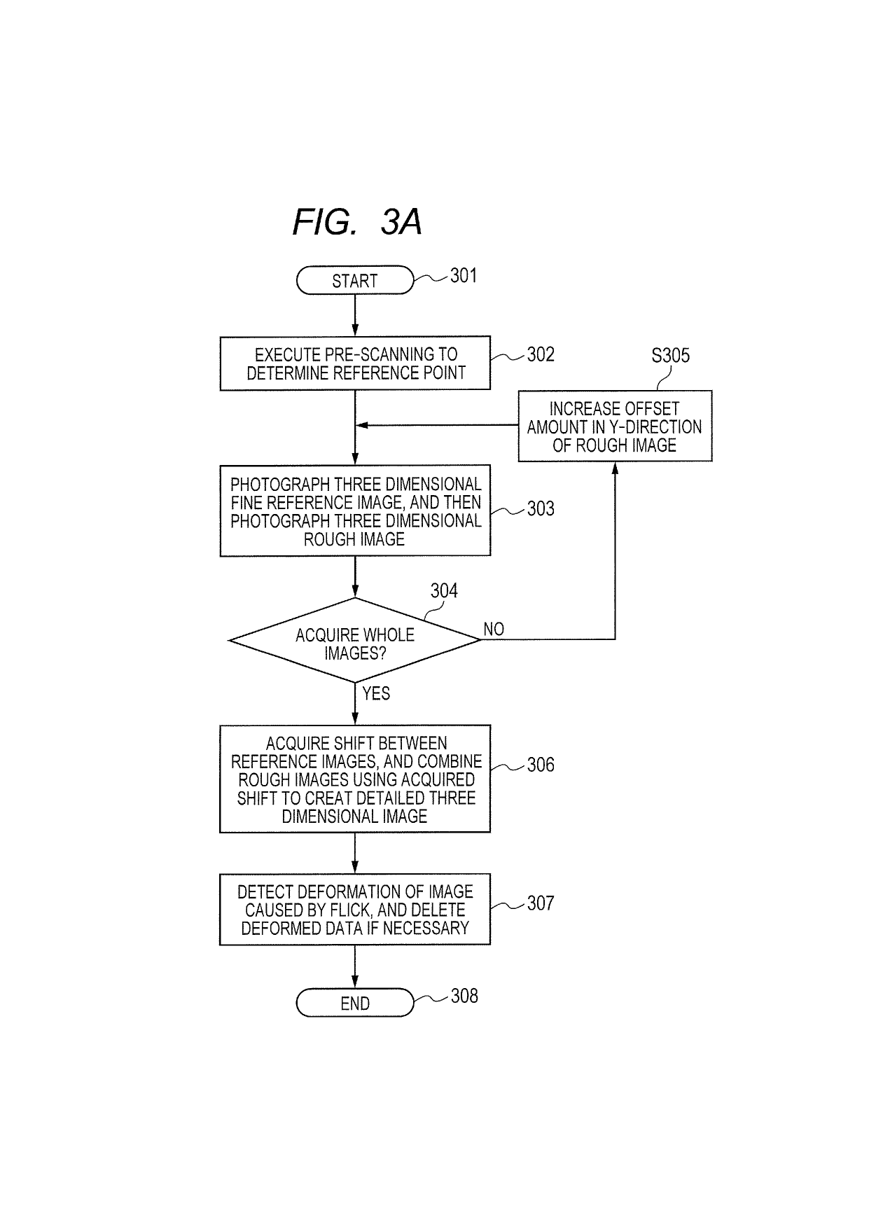

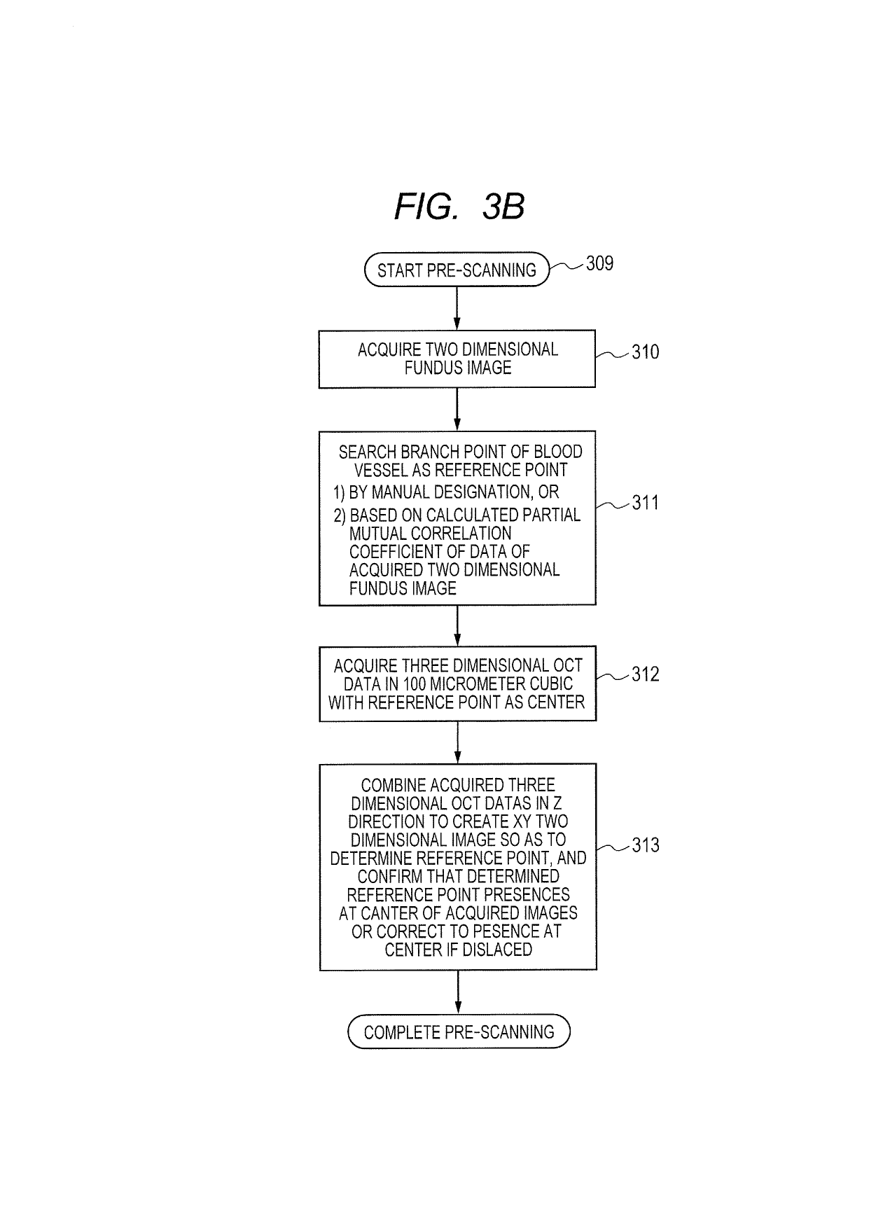

An operation of the present invention to be performed in the OCT apparatus described above is described following a flowchart illustrated in FIG. 3A. In this embodiment, 400 images of B-scan each including 400 dots in the X direction and 400 dots in the Y direction are acquired as described in the Background of the Invention.

When starting imaging in Step 301, pre-scanning is first performed in Step 302 to determine a reference point. Details of the pre-scanning are described in a flowchart illustrated in FIG. 3B. A two-dimensional fundus image is acquired in Step 310 of the flowchart illustrated in FIG. 3B, and a branch point of the blood vessel such as the area 204 illustrated in FIG. 2 is searched and the searched branch point is set as a reference point 204 in Step 311.

As described above, there are similar blood vessels at substantially the same position of the fundus regardless of individual, and therefore, the reference point 204 can be designated manually or set automatically. A method of manually designating the reference point 204 generally includes displaying an image on a display unit and designating the reference point 204 with a pointing device such as a mouse. A method of automatically searching or setting the reference point 204 includes acquiring a partial mutual correlation coefficient of the acquired two-dimensional fundus image and graphic data of the blood vessel of the reference point 204 and setting a point having the largest correlation coefficient as the reference point.

Designating or setting the reference point 204 corresponds to a step of setting a characteristic portion in the present invention. This setting step is executed via a module area functioning as a setting unit in the CPU 110.

When XY coordinates of the reference point is determined, an OCT image (three-dimensional image) around the reference point 204 is acquired in Step 312.

As an area to be acquired, there is acquired a three-dimensional image of the OCT of the reference point in a space equal to or larger than a range of movement of the eye, which is estimated during acquisition of a detailed three-dimensional image. The movement of the eye is at most about 15 minutes of arc, and hence, assuming that an eye axis length is 25 mm, the following expression is obtained:

25 mm×tan(15′)=100 μm

Therefore, the space for acquiring the image has a size of 100 μm in the X, Y, and Z directions. A thickness of the blood vessel is about 30 μm at a narrow branched portion, and hence it is thick enough to include the branch point.

The area from which the three-dimensional image is acquired corresponds to a first area in a method invention and a predetermined area including the characteristic portion in an apparatus invention.

There is an individual difference in a position of the blood vessel and a position of the optic disk in the fundus or wavefront aberration of an anterior eye portion and a shape of the eye. Therefore, depending on the examinee, there occurs a shift in a focusing position of the measuring light and an imaging position of the line sensor or the like. For this reason, as described above, it is preferred to acquire an image of the eye to be inspected by the pre-scanning in advance and then to designate, based on the image, the reference point 204 serving as the characteristic portion that is a portion of the eye to be inspected. As the reference point 204, an area of the eye to be inspected in which the blood vessels are crossed with each other or branched is suitable in selecting the point by a visual contact. The image can be also acquired as a tomographic image (a so-called “image of C-scan”) in the X and Y directions of the fundus by using a fundus camera or an SLO apparatus used together with the OCT apparatus.

Subsequently, in Step 313, correction is performed so that the reference point 204 acquired in Step 311 becomes the center of the OCT image. More specifically, a two-dimensional image is created by superimposing data of the three-dimensional image acquired in Step 312 in the Z axis direction, and the determination of the coordinates of the reference point 204 is performed, which is acquired by a method similar to the method of Step 311. When the reference point 204 is not the center of the acquired image, a position of starting the acquisition of the image data is corrected to locate the reference point 204 at the center of the image. A correction value for the correction can also be used for position correction of an image acquiring unit and the OCT apparatus when acquiring the two-dimensional image.

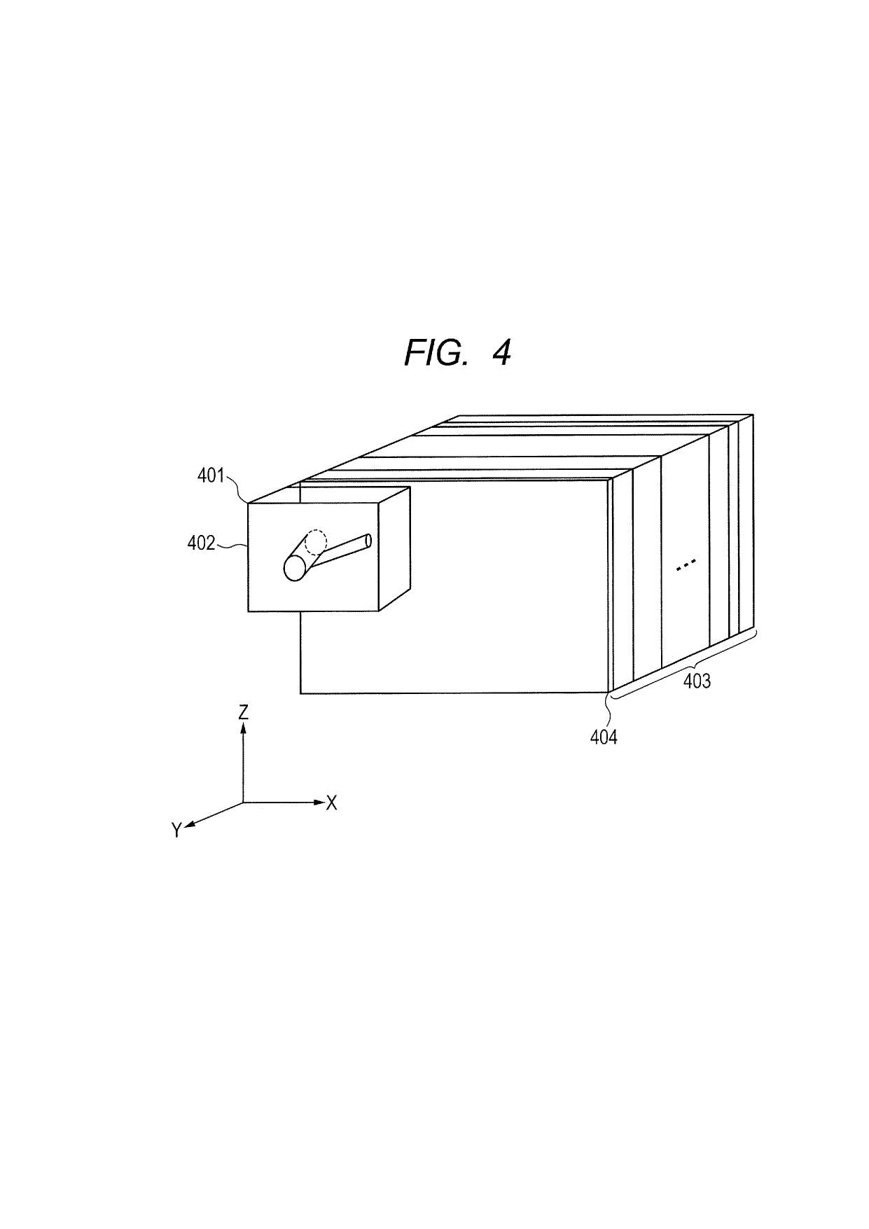

After that, in Step 303, a rough three-dimensional image is acquired. Firstly, an image of the reference determined in Step 302 is acquired, and a rough three-dimensional image of a target portion, for example, the macula, is acquired from a two-dimensional fundus image of an area other than the predetermined area from which the image of the reference is acquired. The acquired image has a configuration as illustrated in FIG. 4. In FIG. 4, a predetermined or first area 401 is a space for acquiring the reference point, and a branch point 402 of the blood vessel is the reference point 204. A plane 403 is a plane for acquiring a two-dimensional tomographic image, which is, in this example, 25 images that can be acquired within 0.1 sec and generally acquired at equal intervals in the Y direction. An offset 404 is an offset of the rough image acquired by the Y coordinate in the Y direction at a plane from which the first two-dimensional tomographic image is acquired. This Y coordinate is shifted (offset) by a predetermined amount, and the images are acquired at equal intervals as the plane 403, with the result that images of different coordinates can be acquired.

The step of acquiring a first three-dimensional image of the first area including the characteristic portion and the tomographic image of a second area different from the first area, i.e., an area that does not include the predetermined area including the characteristic portion, corresponds to a first acquiring step in the present invention. It is preferred that the second area include at least one of the optic disk and the macula that can be easily supplemented by a visual contact. Further, it is preferred that a period of time required for the first acquiring step be within a period of time for which the eye to be inspected performs a fixation fine movement within a predetermined distance, and the period of time is, in this embodiment, 0.1 sec as described above. In addition, it is preferred to set a predetermined amount of the offset as appropriate, and in this embodiment, an offset amount of 15 tomographic images is set because 16 sets of images are needed to acquire 400 tomographic images by repeating a plurality of times the image acquiring step of acquiring the three-dimensional image of the predetermined area including the characteristic portion and 25 tomographic images within a predetermined period. It is preferred that the image forming apparatus according to this embodiment include a tomographic image acquiring unit for executing the first acquiring step and a second acquiring step described later. The tomographic image acquiring unit has, for example, a function of receiving tomographic image data transferred from the outside.

The predetermined amount can be set arbitrarily in accordance with an amount of the image set, can be a fixed amount, and further can be changed automatically in accordance with a size of an area that is taken as an image acquiring range. Moreover, details of the image set can differ from one another by causing periods of time for acquiring the images in a plurality of image acquiring steps to differ from one another. In this case, a period of time required to acquire the first image set is a first predetermined period, a period of time required to acquire another image set is a second predetermined period, and each of the acquiring steps is performed within each of the periods of time. An instruction for executing the above-mentioned image acquiring step with respect to the OCT apparatus is issued by a module area functioning as an image acquisition instructing unit in the CPU 110.

Subsequently, in Step 304, when the 16 sets of rough images of target detailed images are acquired, the process control proceeds to Step 306, and otherwise, the offset 404 is shifted by one pixel in Step 305, and the image is acquired in Step 303. This operation corresponds to, in the present invention, the second acquiring step of acquiring a second three-dimensional image in the first area within the second predetermined period, and acquiring a second tomographic image that is a part of the tomographic image different from the first tomographic image acquired in advance among a plurality of tomographic images in the second area.

After that, in Step 306, the plurality of rough images acquired in Step 303 are combined. A method of combining the rough images includes setting, as a reference, the characteristic portion that is the reference point 204 of the first acquired image, acquiring a difference of the coordinates between the reference and the reference point of the target image, subtracting the acquired difference of the coordinates from coordinates of the target image, and combining the image with the first image. That is, the characteristic portions in the above-mentioned first and second three-dimensional images are set as references, alignment of the first and second tomographic images is performed based on a shift therebetween, and then the images are combined. By repeating these processes, the difference of the coordinates of the rough images due to a vibration of the eye is removed, and a fine three-dimensional image can be acquired. This operation is executed by a module area in the CPU 110, which functions as an image combining unit in the present invention. A composite image acquired by aligning the first and second three-dimensional images is displayed on the monitor serving as the display unit by a display control unit that designates an image to be displayed on the display unit.

A method of acquiring the difference of the coordinates between two reference points includes a method of acquiring coordinates of an intersection of intersecting blood vessels and a method of least squares in which such Δx, Δy, and Δz are acquired that a value of the following expression is minimized:

$${\sum }_{Z = 0}^{400} {\sum }_{Y = 0}^{400} {\sum }_{X = 0}^{400} {( A ( X , Y , Z ) - B ( X - Δ x , Y - Δ y , Z - Δ z ) )}^{2}$$

where A(X,Y,Z) and B(X,Y,Z) represent image brightness in the tomographic images.

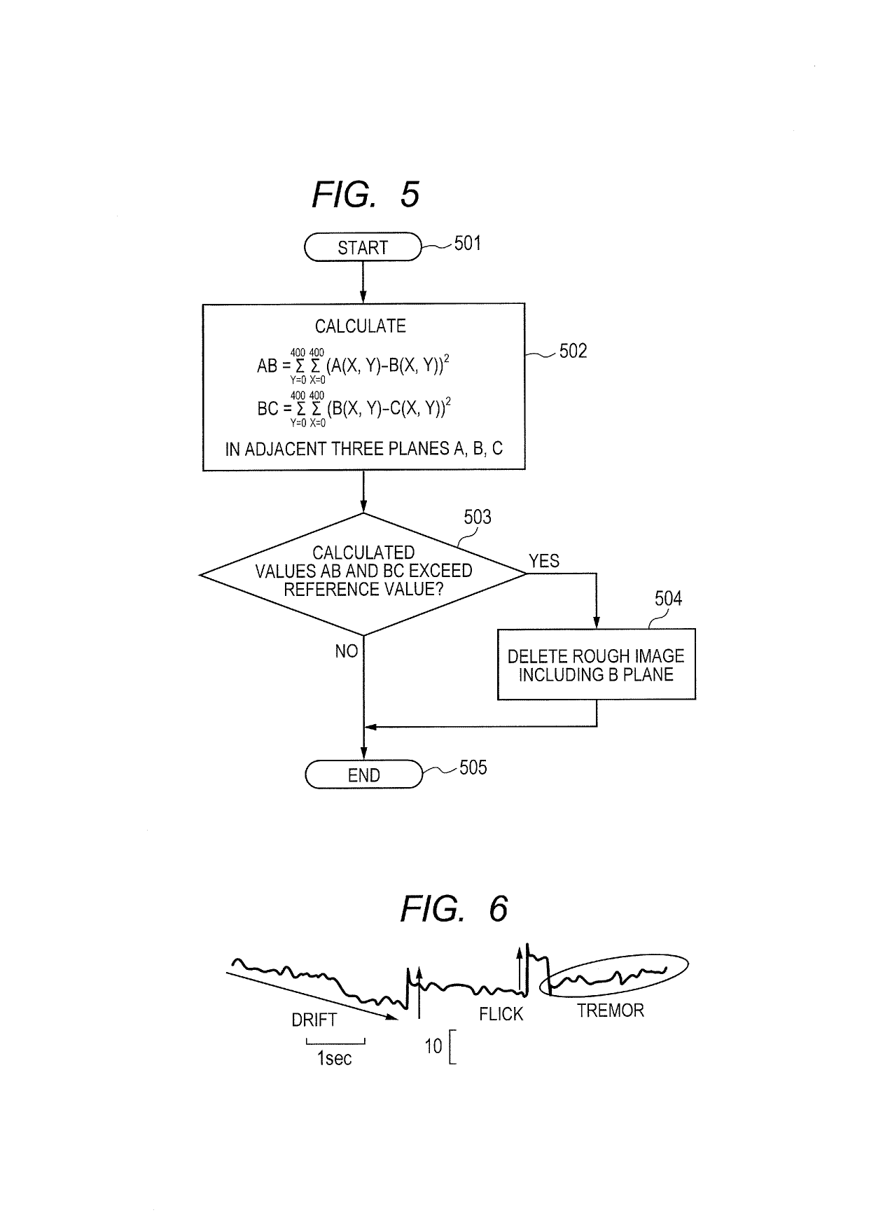

Subsequently, in Step 307, distortion of the image due to the flick is detected as described above. As illustrated in FIG. 6, the flick is a large movement but its cycle is as long as several seconds, and hence one or no flick is included in a single imaging operation. In order to detect the flick, it suffices to detect whether or not there is a shift of the coordinates between start and end of each imaging operation. A start plane is fixed at the reference point, and hence it suffices to detect whether or not an end plane is shifted from the other planes. FIG. 5 is a flowchart illustrating an evaluation on a plane. The process starts in Step 501. In Step 502, a sum of squares of a brightness difference between acquired images is acquired with respect to adjacent planes A, B, and C. The acquired values are set as AB and BC, respectively. That is, when a tomographic image among the above-mentioned first tomographic images and a tomographic image among the above-mentioned second tomographic images are adjacent to each other, a difference of the brightness between the tomographic images is acquired.

In Step 503, when it is determined that the two values are equal to or less than a reference value, it can be considered that there is no significant change between the two planes. On the other hand, when it is determined that the two values exceed the reference value, there is a risk that one of the planes represents an image of a place shifted by the flick. From this point, when the sum of squares of the difference of the brightness exceeds the reference value with respect to two adjacent planes, it can be considered that only one of the planes is abnormal. Therefore, when the evaluation is “YES” in this step, in Step 504, all the rough images including the plane B are deleted. Regarding a plane on an edge, there is only one adjacent plane, and hence, when it is determined that the adjacent plane is normal through the determination that the value exceeds the reference value, it can be determined that the plane on the edge is abnormal.

By performing the above-mentioned operation for the last 16 planes of each of the rough images, the distortion due to the flick can be removed.

When all the rough images are deleted, the deleted pieces of data are missing, and therefore, a more flawless three-dimensional image can be acquired by re-acquiring rough images of the same positions.

Another method of detecting the distortion of the image due to the flick includes a method of re-acquiring the image of the area acquired in Step 302 after once acquiring the rough images in the Y direction. That is, after acquiring the first three-dimensional image within the first predetermined period, a third three-dimensional image is acquired in the first area within the first predetermined period, and an amount of position shift between the first and third three-dimensional images is acquired. In other words, when the reference points at the start and the end of the imaging are compared and it is determined that a difference or a shift amount of the reference points exceeds a threshold value as the reference value, i.e., when it is determined that the reference point is shifted in a large amount, it can be considered that the flick is generated. In this case as well, a more flawless three-dimensional image can be acquired by re-acquiring the rough images in the same Y direction.

Alternatively, data that can be acquired from the deleted tomographic image can be interpolated from images adjacent to the deleted tomographic image. With this operation, even when the flick is generated at the time of imaging, a composite image can be generated in a shorter period of time than in the case of re-acquiring the images.

In addition to the case of repeating a plurality of times the acquisition of the above-mentioned three-dimensional image and tomographic image, for example, a mode for acquiring at one time tomographic images of an area other than a predetermined area for the purpose of acquiring a normal tomographic image can be added. In this case, it is preferred to further provide a selecting unit that causes the OCT apparatus to select one of the mode for repeating the acquiring step a plurality of times or the mode for acquiring the images of the area other than the predetermined area at one time, and to execute the selected mode via the image acquisition instructing unit.

### Other Embodiments

In addition, the present invention can be realized also by performing the following process. Specifically, software (program) for realizing the functions of the above-mentioned embodiments is supplied to a system or an apparatus via a network or various storage media, and a computer (or CPU, MPU, or the like) of the system or the apparatus reads and executes the program.

In the above-mentioned embodiment, a case of targeting an eye to be inspected as an object to be inspected is described. However, as described above, the present invention is not limited to any particular object, but, for example, the object to be inspected can be a skin or an organ of an examinee. In this case, the present invention can be applied to a medical apparatus such as an endoscope in addition to the ophthalmological apparatus.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-006007, filed Jan. 16, 2012, which is hereby incorporated by reference herein in its entirety.

What is claimed is:

1. An image forming method of acquiring a plurality of tomographic images of an object to be inspected by using an optical coherence tomographic method and forming a three-dimensional image of the object based on the plurality of acquired tomographic images, the image forming method comprising:
acquiring, within a first predetermined period, a first three-dimensional image of a first area including a characteristic portion of the object and first tomographic images as a part of the plurality of tomographic images of a second area different from the first area;
acquiring, within a second predetermined period, a second three-dimensional image of the first area and second tomographic images as a part of the plurality of tomographic images of the second area, the second tomographic images being different from the first tomographic images; and
aligning positions of the first tomographic images and the second tomographic images by using, as references, the characteristic portion included in the first three-dimensional image and the characteristic portion included in the second three-dimensional image.

2. An image forming method according to claim 1, wherein:
the object comprises an eye to be inspected; and
the first predetermined period and the second predetermined period comprise a period for which the eye performs a fixation fine movement within a predetermined distance.

3. An image forming method according to claim 2, wherein:
the second area comprises at least one of an optic disk and a macula of the eye; and
the characteristic portion comprises one of a portion in the eye where blood vessels intersect with each other and a portion in the eye where a blood vessel is branched.

4. An image forming method according to claim 1, further comprising selecting, after acquiring a three-dimensional image of the object, a portion of the object included in the three-dimensional image as the characteristic portion.

5. An image forming method according to claim 1, further comprising:
acquiring, when a single tomographic image among the first tomographic images and a single tomographic image among the second tomographic images are adjacent to each other, a difference of brightness between the single tomographic images; and
re-acquiring the second tomographic images when the difference exceeds a threshold value.

6. An image forming method according to claim 1, further comprising:
acquiring a third three-dimensional image of the first area within the first predetermined period after acquiring the first three-dimensional image within the first predetermined period;
acquiring a shift amount of positions of the first three-dimensional image and the third three-dimensional image; and
re-acquiring the second tomographic images when the shift amount exceeds a threshold value.

7. An image forming method according to claim 1, further comprising displaying, on a display unit, an image obtained by aligning the positions of the first tomographic images and the second tomographic images.

8. An image forming apparatus, comprising an image forming portion for executing the image forming method according to claim 1.

9. A recording medium for storing a program for causing a computer to execute the steps of the image forming method according to claim 1.

10. An image forming apparatus for acquiring a plurality of tomographic images of an object to be inspected by using an optical coherence tomographic method and forming a three-dimensional image of the object based on the plurality of acquired tomographic images, the image forming apparatus comprising:
a tomographic image acquiring unit configured to:
acquire, within a first predetermined period, a first three-dimensional image of a first area including a characteristic portion of the object and first tomographic images as a part of the plurality of tomographic images of a second area different from the first area; and
acquire, within a second predetermined period, a second three-dimensional image of the first area and second tomographic images as a part of the plurality of tomographic images of the second area, the second tomographic images being different from the first tomographic images; and
a unit configured to align positions of the first tomographic images and the second tomographic images by using, as references, the characteristic portion included in the first three-dimensional image and the characteristic portion included in the second three-dimensional image.

11. An image forming apparatus according to claim 10, wherein:
the object comprises an eye to be inspected; and
the first predetermined period and the second predetermined period comprise a period for which the eye performs a fixation fine movement within a predetermined distance.

12. An image forming apparatus according to claim 11, wherein:
the second area comprises at least one of an optic disk and a macula of the eye; and
the characteristic portion comprises one of a portion in the eye where blood vessels intersect with each other and a portion in the eye where a blood vessel is branched.

13. An image forming apparatus according to claim 11, further comprising a display control unit configured to display, on a display unit, an image obtained by aligning the positions of the first tomographic images and the second tomographic images.

14. An image forming system for acquiring a plurality of tomographic images of an object to be inspected by using an optical coherence tomographic method and forming a three-dimensional image of the object based on the plurality of acquired tomographic images, the image forming system comprising:
an optical coherence tomography (OCT) apparatus configured to acquire a three-dimensional image of the object;
an image acquisition instructing unit configured to:
cause the OCT apparatus to execute, within a predetermined period, acquisition of a three-dimensional image of a predetermined area including a characteristic portion of the object and acquisition of tomographic images as a part of the plurality of tomographic images of an area other than the predetermined area; and
cause the OCT apparatus to repeat the acquisition of the three-dimensional image and the acquisition of the tomographic images a plurality of times; and
an image combining unit configured to combine the plurality of tomographic images based on a position shift of the three-dimensional images of the characteristic portion, which are obtained by repeating the acquisition of the three-dimensional image and the acquisition of the tomographic images a plurality of times.

15. An image forming system according to claim 14, wherein:
the object comprises an eye to be inspected; and
the predetermined period comprises a period for which the eye performs a fixation fine movement within a predetermined distance.

16. An image forming system according to claim 15, wherein:
the area other than the predetermined area comprises at least one of an optic disk and a macula of the eye; and
the characteristic portion comprises one of a portion in the eye where blood vessels intersect with each other and a portion in the eye where a blood vessel is branched.

17. An image forming system according to claim 14, further comprising a display control unit configured to display, on a display unit, an image obtained by aligning positions of the plurality of tomographic images.

18. An image forming system according to claim 14, further comprising a selecting unit configured to:
select, as a mode for causing the OCT apparatus to acquire the tomographic images, any one of a mode for repeating the acquisition of the three-dimensional image and the acquisition of the tomographic images a plurality of times and a mode for acquiring the tomographic images of the area other than the predetermined area at one time; and
cause the OCT apparatus to execute the selected mode via the image acquisition instructing unit.
